(19) United States
(12) Patent Application Publication
(10) Pub. No.: US 2026/0137824 A1
(43) Pub. Date: May 21, 2026

(54) Adjustable Sterilization Shelf

(71) Applicant: METAL INDUSTRIES RESEARCH & DEVELOPMENT CENTRE, Kaohsiung City (TW)

(72) Inventors: KUAN-YU CHIU, Chiayi Country (TW); CHUN-CHIEH TSENG, Kaohsiung City (TW); BANG-SIA CHEN, Kinmen County (TW); LING-ZHEN KAO, Tainan City (TW)

(21) Appl. No.: 18/954,562

(22) Filed: Nov. 21, 2024

(51) Int. Cl. A61L 2/26; A47B 57/04; A47B 57/30; A61L 2/20

(52) U.S. Cl. CPC A61L 2/26; A47B 57/20; A61L 2/20; A61L 2103/15; A61L 2202/15

(57) ABSTRACT

An adjustable sterilization shelf is provided to solve the problems of limited space and poor sterilization effect encountered by conventional sterilization devices. The adjustable sterilization shelf includes a frame and at least one adjustable board. The frame includes a plurality of posts disposed upright and around a sterilization space. The at least one adjustable board is disposed in the sterilization space. The at least one adjustable board is slidable in a vertical direction and is coupled with the plurality of posts. Each of two opposite sides of the at least one adjustable board is provided with a flow guiding member. Each flow guiding member includes a flow guiding face facing the sterilization space. The flow guiding face is at a first inclination angle to a surface of the at least one adjustable board. The first inclination angle is greater than 90 degrees.

### BACKGROUND OF THE INVENTION

### 1. Field of the Invention

The present invention relates to a medical device and, more particularly, to an adjustable sterilization shelf which has a receiving space adjustable according to the size of an object to be sterilized and which enables uniform distribution of the sterilizing gas.

### 2. Description of the Related Art

The aseptic quality of medical devices must be strictly controlled according to the usage condition, the contact area, and the usage time to reduce the infection risks in the medical treatment. Common sterilization methods include radiation sterilization, moist heat sterilization, and gas sterilization. High temperature and ultraviolet light will cause deterioration of devices made of certain plastic materials. Therefore, gases, such as ethylene oxide (EO) or oxygen nitride (NO₂), may be distributed in a sterilization space to proceed with sterilization of medical devices made of most materials.

Since the size of the sterilization space of the above conventional gas sterilization devices for receiving to-be-sterilized objects is fixed, the stacking pattern and placement position of the to-be-sterilized objects are restricted. Furthermore, the to-be-sterilized objects have various sizes and shapes and, thus, may form dead spaces in the sterilization space, such that the sterilizing gas cannot uniformly flow to all corners of the sterilization space, thereby failing to achieve thorough sterilization.

Thus, it is necessary to improve the conventional sterilization devices.

### BRIEF SUMMARY OF THE INVENTION

To solve the above problem, it is an objective of the present invention to provide an adjustable sterilization shelf having a receiving space which may be adjusted according to the sizes and the shapes of the objects received in the receiving space.

It is another objective of the present invention to provide an adjustable sterilization shelf permitting uniform placement of the objects to be sterilized.

It is a further objective of the present invention to provide an adjustable sterilization shelf which may increase the uniformity of distribution of the sterilizing gas.

It is still another objective of the present invention to provide an adjustable sterilization shelf which may enhance the sterilization efficiency and the sterilization reliability.

As used herein, the term “a”, “an” or “one” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

As used herein, the term “engagement”, “coupling”, “assembly”, or similar terms is used to include separation of connected members without destroying the members after connection or inseparable connection of the members after connection. A person having ordinary skill in the art would be able to select according to desired demands in the material or assembly of the members to be connected.

An adjustable sterilization shelf according to the present invention includes a frame and at least one adjustable board. The frame includes a plurality of posts disposed upright and around a sterilization space. The at least one adjustable board is disposed in the sterilization space. The at least one adjustable board is slidable in a vertical direction, namely, the extending direction of the plurality of posts, and is coupled with the plurality of posts. Each of two opposite sides of the at least one adjustable board is provided with a flow guiding member. Each flow guiding member includes a flow guiding face facing the sterilization space. The flow guiding face is at a first inclination angle to a surface of the at least one adjustable board. The first inclination angle is greater than 90 degrees.

Thus, in the adjustable sterilizing shelf according to the present invention, the sliding members slidable in the vertical direction enables adjustment of the height of each adjustable board, such that the receiving space between two adjustable boards may be selected according to the size and the shape of the object to be sterilized. Furthermore, each adjustable board is surrounded by the plurality of flow guiding members, such that the sterilizing gas may be guided to circulate in the space between each two adjacent adjustable boards to thereby increase the distribution uniformity of the sterilizing gas.

In an example, the at least one adjustable board is provided with at least one pair of sliding members. Each sliding member includes at least one sliding portion which is slidable in the vertical direction and which is coupled with a respective one of the plurality of posts. Each sliding member includes a supporting face. The supporting faces of the at least one pair of sliding members are located on a same horizontal plane. The at least one adjustable board is located on the supporting faces of the at least one pair of sliding members. Thus, the height of each sliding member may be selected to locate the supporting faces on the same height to place the board horizontally, thereby enabling adjustment of the height of the shelf and the size of the space.

In an example, each flow guiding member is coupled with the supporting faces of the at least one pair of sliding members. Thus, each flow guiding member may be located on the same height as the board, thereby guiding the gas currents to achieve uniform distribution on the board.

In an example, a lower surface of each sliding member is provided with an auxiliary flow guiding member. Each auxiliary flow guiding member includes an auxiliary flow guiding face facing the sterilization space. The auxiliary flow guiding face is at a second inclination angle to a lower surface of the at least one adjustable board. The second inclination angle is greater than 90 degrees. Thus, each flow guiding member may guide gas currents to flow toward other boards in the sterilization space, thereby increasing the convention effect.

In an example, each of the plurality of posts includes a plurality of positioning portions spaced from each other in the vertical direction. Each sliding member includes a coupling portion securely coupled in one of the plurality of positioning portions. Thus, the positioning portions at different heights permit selection of the height of the board, thereby enabling adjustment and fixing of the position of the board.

In an example, each sliding member includes a support and a pin. Each of two ends of the support forms one of the sliding portions. Each sliding portion includes a receiving hole slidably receiving a respective one of the plurality of posts. Each of two ends of the pin forms one of the coupling portions. The two coupling portions are associated with the two sliding portions. Each coupling portion extends through a lateral hole of a respective one of the sliding portions and is then securely coupled with one of the plurality of positioning portions of a respective one of the plurality of posts. Thus, each sliding member is slidable in the vertical direction and may be used to determine the fixed height of the supporting face of the sliding member by selecting the height of the positioning portion into which the coupling portion is inserted, thereby enabling adjustment of the height of the shelf and the size of the space.

In an example, each sliding member is provided with an elastic element. Two ends of the elastic element respectively abut against a fixing portion of the support and a force application portion of the pin. Thus, when the support and the pin are moved relative to each other, the elastic element may generate a returning force to push the sliding member back to the fixing state after adjustment of the height, thereby enabling fixing of the height of the sliding member.

In an example, the pin includes two sections assembled with each other at a junction. Each of two ends respectively of the two sections remote from the junction includes one of the coupling portions. The two sections are engaged with the fixing portion of the support. The elastic element is held between the fixing portion of the support and the force application portion of the pin. Thus, when the pin is assembled, the two coupling portions are respectively engaged with the two sliding portions while assembling the support and the elastic element, thereby enabling convenient assembly.

In an example, the flow guiding face of each flow guiding member faces inward toward the sterilization space. Each flow guiding member further includes a flow inducing face opposite to the flow guiding face. The cross section of each flow guiding member is an approximate isosceles triangle and has two sides. Each of the two sides is concave, and the two sides respectively form the flow inducing face and the flow guiding face. Thus, the streamlined curved face may reduce the wind resistance and guide the flowing direction, thereby increasing gas flowability.

In another example, the flow guiding face of each flow guiding member faces inward toward the sterilization space. Each flow guiding member further includes a flow inducing face opposite to the flow guiding face. The cross section of each flow guiding member is in a form of a fish fin. The fish fin includes a wavy face forming the flow inducing face and a concave face forming the flow guiding face. Thus, the guiding members enable concentration and circumfluence of the gas, thereby avoiding the gas from staying in the dead spaces or uneven distribution.

In an example, the at least one adjustable board is in the form of a mesh board. Thus, when gas currents circulate between two adjacent boards, the gas currents may flow into other spaces of the shelf, thereby increasing the dispersion uniformity of the gas.

### DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is an exploded, perspective view of an adjustable sterilization shelf of a preferred embodiment according to the present invention.

FIG. 2 is an exploded, perspective view of a sliding member of the adjustable sterilization shelf of the preferred embodiment according to the present invention.

FIG. 3 is a perspective view of the adjustable sterilization shelf of the preferred embodiment according to the present invention after assembly.

FIG. 4 is a cross sectional view taken along section line 4-4 of FIG. 3.

FIG. 5 is an enlarged view of a circled portion V of FIG. 4.

FIG. 6 is a view similar to FIG. 5, illustrating another embodiment of the adjustable sterilization shelf.

FIG. 7 is a cross sectional view taken along section line 7-7 of FIG. 4.

FIG. 8 is a view similar to FIG. 7, illustrating movement of the sliding member to permit adjustment in the height.

FIG. 9 is a view similar to FIG. 4, illustrating another embodiment of the adjustable sterilization shelf.

FIG. 10 is an enlarged view of a circled portion X of FIG. 9.

When the terms “front”, “rear”, “left”, “right”, “up”, “down”, “top”, “bottom”, “inner”, “outer”, “side”, and similar terms are used herein, it should be understood that these terms have reference only to the structure shown in the drawings as it would appear to a person viewing the drawings and are utilized only to facilitate describing the invention, rather than restricting the invention.

### DETAILED DESCRIPTION OF THE INVENTION

In order to make the above and other objectives, features, and advantages of the present invention clearer and easier to understand, preferred embodiments of the present invention will be described hereinafter in connection with the accompanying drawings. Furthermore, the elements designated by the same reference numeral in various figures will be deemed as identical, and the description thereof will be omitted.

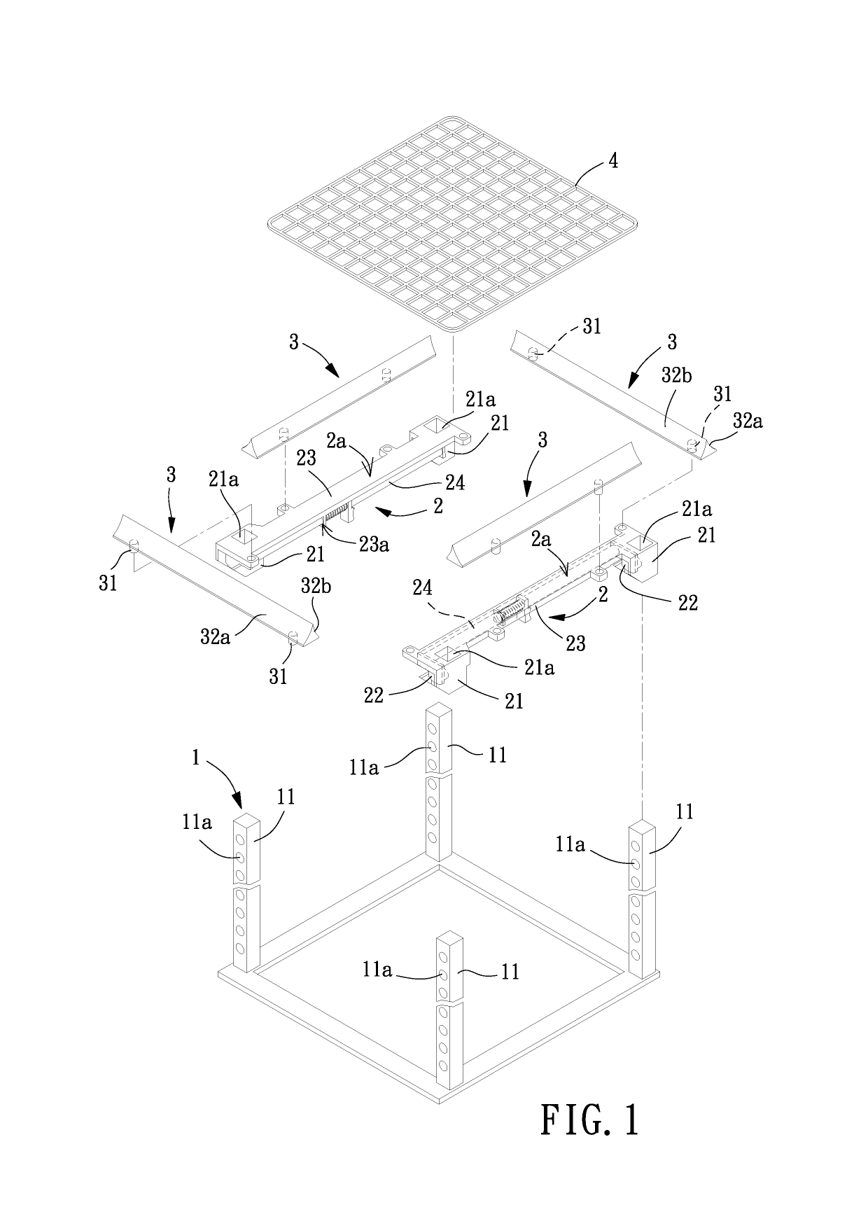

With reference to FIG. 1, an adjustable sterilization shelf of a preferred embodiment according to the present invention includes a frame 1, at least one pair of sliding members 2, a plurality of flow guiding members 3, and at least one adjustable board 4. The at least one pair of sliding members 2 is disposed on the frame 1. The sliding members 2 of the same pair may slide along the frame 1 to the same height through adjustment. The plurality of flow guiding members 3 is located on the at least one pair of sliding members 2. The at least one adjustable board 4 is located on the sliding members 2 of the same pair. The plurality of flow guiding members 3 is placed around the at least one adjustable board 4.

The frame 1 includes a plurality of posts 11 disposed upright and around a sterilization space. Each of the plurality of posts 11 includes a plurality of positioning portions 11a spaced from each other in the vertical direction at regular intervals. Furthermore, the height of the positioning portions 11a on each of the plurality of posts 11 correspond to those of the positioning portions 11a on other posts 11. In this embodiment, the frame 1 includes four posts 11 respectively on four corners of a rectangle. Each positioning portion 11a may be in the form of a hole to permit insertion of an object, thereby fixing at the height of a respective positioning portion 11a. Nevertheless, each positioning portion 11a may be in the form of a protrusion to couple with another object. The present invention is not limited to the quantity of the posts 11 and the coupling mechanism for each positioning portion 11a.

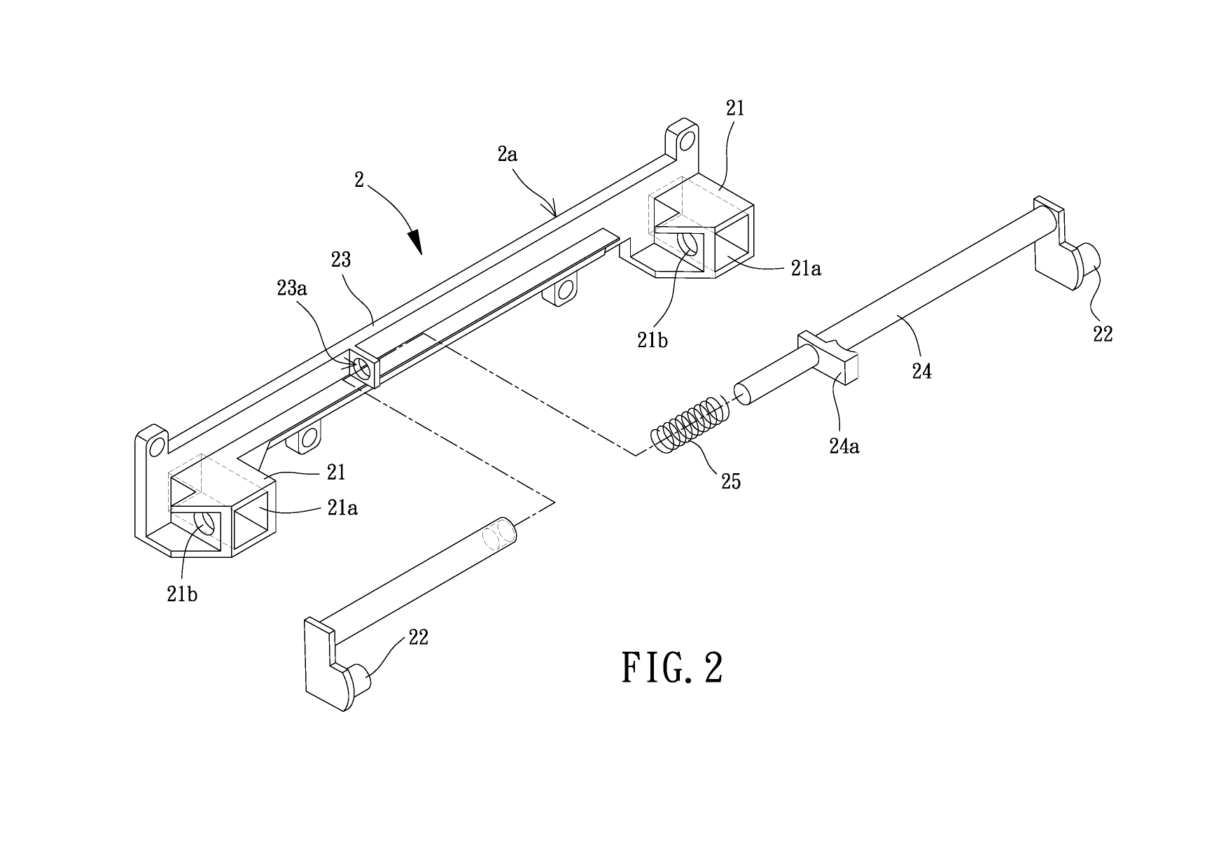

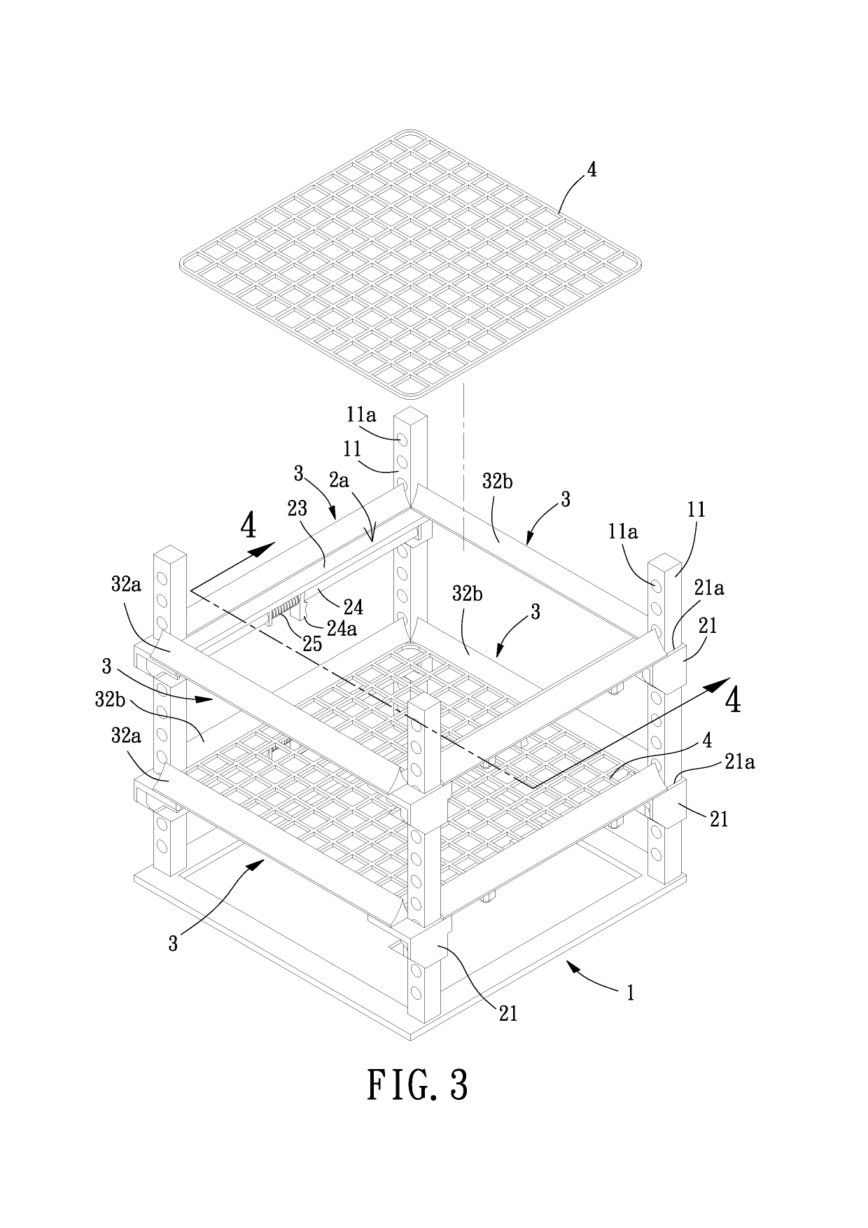

With reference to FIGS. 2 and 3, each sliding member 2 includes at least one sliding portion 21 which is slidable in the vertical direction and which is coupled with a respective one of the plurality of posts 11. After moving to the selected height, each sliding portion 21 may be coupled with a respective one of the positioning portions 11a via an engaging portion 22, such that each sliding member 2 is fixed on the respective post 11. Preferably, the sliding members 2 of the same pair are fixed on the same height. Each sliding member 2 may further include a supporting face 2a facing upward. The supporting faces 2a of the sliding member 2 of the same pair are located on the same horizontal plane.

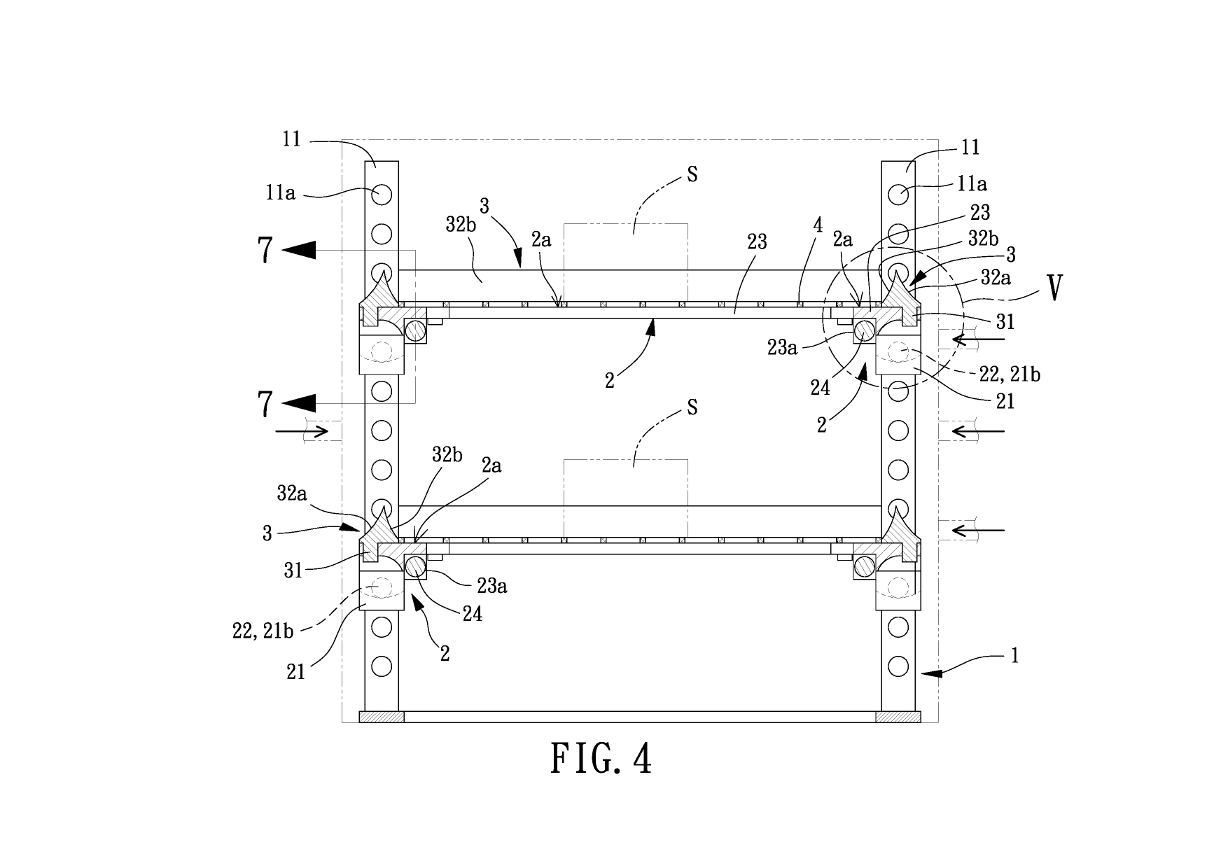

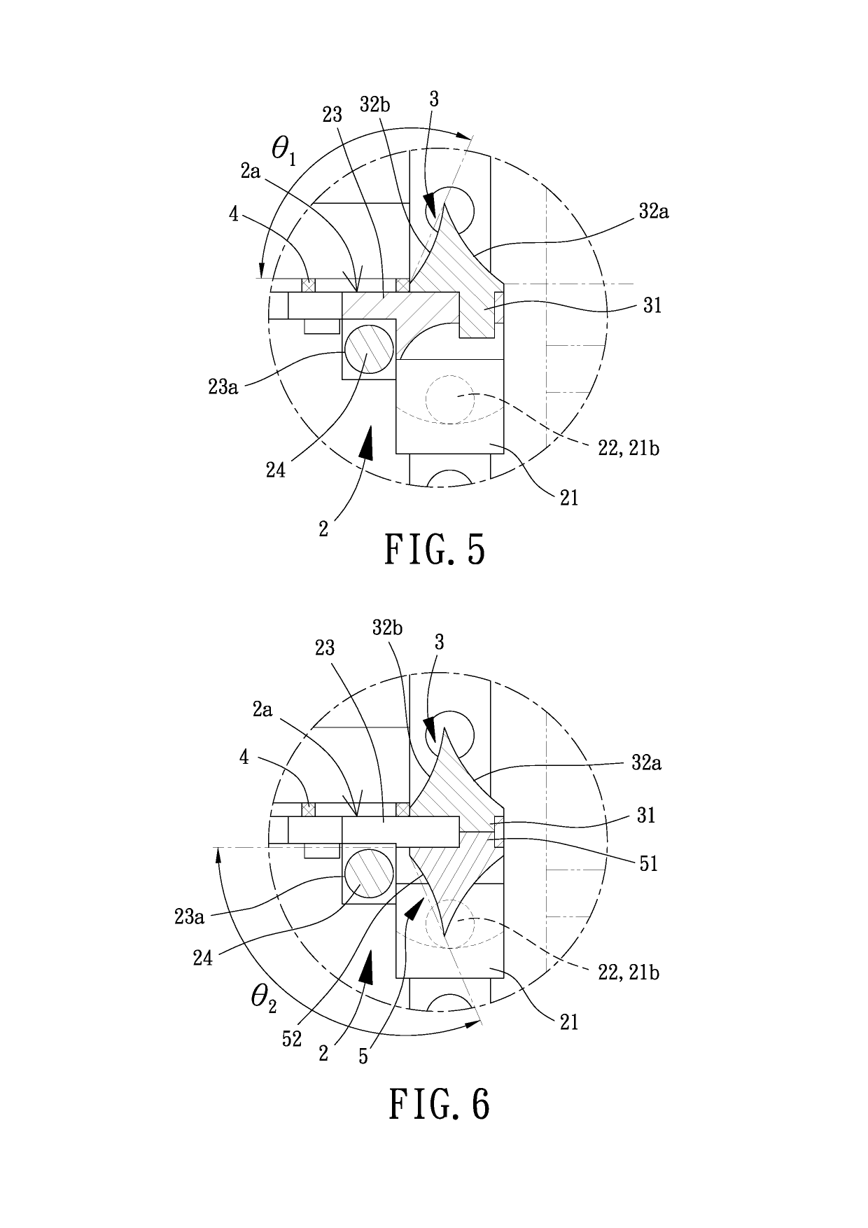

With reference to FIGS. 4 and 5, the plurality of flow guiding members 3 preferably surrounds the frame 1. Each of the plurality of flow guiding members 3 may be located between two adjacent posts 11. The plurality of flow guiding members 3 may be coupled with the supporting faces 2a of the at least one pair of sliding members 2. In this embodiment, each of the plurality of flow guiding members 3 may be coupled with a respective sliding member 2 by two protrusions 31. Alternatively, each of the plurality of flowing guiding members 3 may extend across and couple with the sliding members 2 of the same pair, and four flow guiding members 3 surround the frame 1 to form a rectangular space. Nevertheless, the present invention is not limited to the above coupling mechanism of the flow guiding members 3. Furthermore, as shown in FIG. 5, the cross section of each flow guiding member 3 may be similar to an isosceles triangle and has two sides facing inward and outward, respectively. Each of the two sides is preferably concave. The two sides respectively form the flow inducing face 32a facing outward and the guiding face 32b facing inward (facing the sterilization space). This enables concentration and uniform distribution of gas currents.

The at least one adjustable board 4 is disposed in the sterilization space. The at least one adjustable board 4 is supported on a horizontal plane corresponding to and by the supporting faces 2a of the at least one pair of sliding members 2. The at least one adjustable board 4 is preferably surrounded by the plurality of flow guiding members 3. The flow guiding face 32b of each flow guiding member 3 is at a first inclination angle θ₁ to a surface of the at least one adjustable board 4. The first inclination angle θ₁ is greater than 90 degrees. The at least one adjustable board 4 is used to place objects to be sterilized and may be simply detached and assembled. Furthermore, the height of the at least one adjustable board 4 may be decided by the selected height of the at least one pair of sliding members 2. Therefore, the height difference between two adjacent adjustable boards 4 and the total number of the layers of the shelf may be adjusted according to the sizes and quantity of the to-be-sterilized objects, such that the to-be-sterilized objects may be evenly placed on the at least one adjustable board 4 to avoid squeezing or excessive stacking. Furthermore, the at least one adjustable board 4 is preferably in the form of a mesh board, such that when gas currents circulate between two adjacent adjustable boards 4, the gas currents may flow into other spaces of the shelf through the mesh, thereby increasing the dispersion uniformity of the gas.

With reference to FIG. 6, the adjustable sterilization shelf according to the present invention may further include a plurality of auxiliary flow guiding members 5. Each of the plurality of auxiliary flow guiding members 5 may be coupled with a lower surface of a respective one of the sliding members 2 by a protrusion 51. Furthermore, each of the plurality of auxiliary flow guiding members 5 includes an auxiliary flow guiding face 52 facing the sterilization space. The auxiliary flow guiding face 52 is at a second inclination angle θ₂ to a lower surface of the at least one adjustable board 4. The second inclination angle θ₂ is greater than 90 degrees.

Still referring to FIGS. 2 and 3 showing a preferred embodiment of the sliding member 2, each sliding member 2 includes a support 23. Each of two ends of the support 23 forms one of the sliding portions 21. Each sliding portion 21 includes a receiving hole 21a slidably receiving a respective one of the plurality of posts 11. Each sliding member 2 may further include a pin 24. Each of two ends of the pin 24 forms one of the coupling portions 22. The two coupling portions 22 are associated with the two sliding portions 21. Each coupling portion 22 extends through a lateral hole 21b of a respective one of the sliding portions 21, such that each coupling portion 22 may extend in a lateral direction into a respective receiving hole 21a and is then inserted into one of the plurality of positioning portions 11a of a respective one of the plurality of posts 11. Furthermore, each sliding member 2 is provided with an elastic element 25. Two ends of the elastic element 25 respectively abut against a fixing portion 23a of the support 23 and a force application portion 24a of the pin 24. In this embodiment, the pin 24 is detachably assembled by two sections each having a coupling portion 22. Each coupling portion 22 is remote from the junction where the two sections are engaged with each other. Therefore, the two coupling portions 22 may be coupled with the two sliding portions 21, respectively. Then, the left and right sections of the pin 24 are engaged with the fixing portion 23a of the support 23. At the same time, the elastic element 25 is held between the fixing portion 23a of the support 23 and the force application portion 24a of the pin 24, thereby enabling convenient assembly. Nevertheless, the present invention is not limited to the way the pin 24 engages with the support 23 as illustrated above.

Still referring to FIG. 4, when the sliding members 2 of the same pair are fixed at the same height, an adjustable board 4 may be placed on the supporting faces 2a and is kept horizontal to stably support an object S. The plurality of flow guiding members 3 is fixed to the supporting faces 2a and surround the adjustable board 4 and the object S. Alternatively, as shown in FIG. 6, each of the plurality of auxiliary flow guiding members 5 may be coupled with a lower surface of a respective pair of sliding members 2. By provision of the plurality of flow guiding members 3, the plurality of auxiliary flow guiding members 5, and each adjustable board 4, a sterilizing gas may be guided in a lateral direction into the space between two adjacent boards 4 to sterilize the object S.

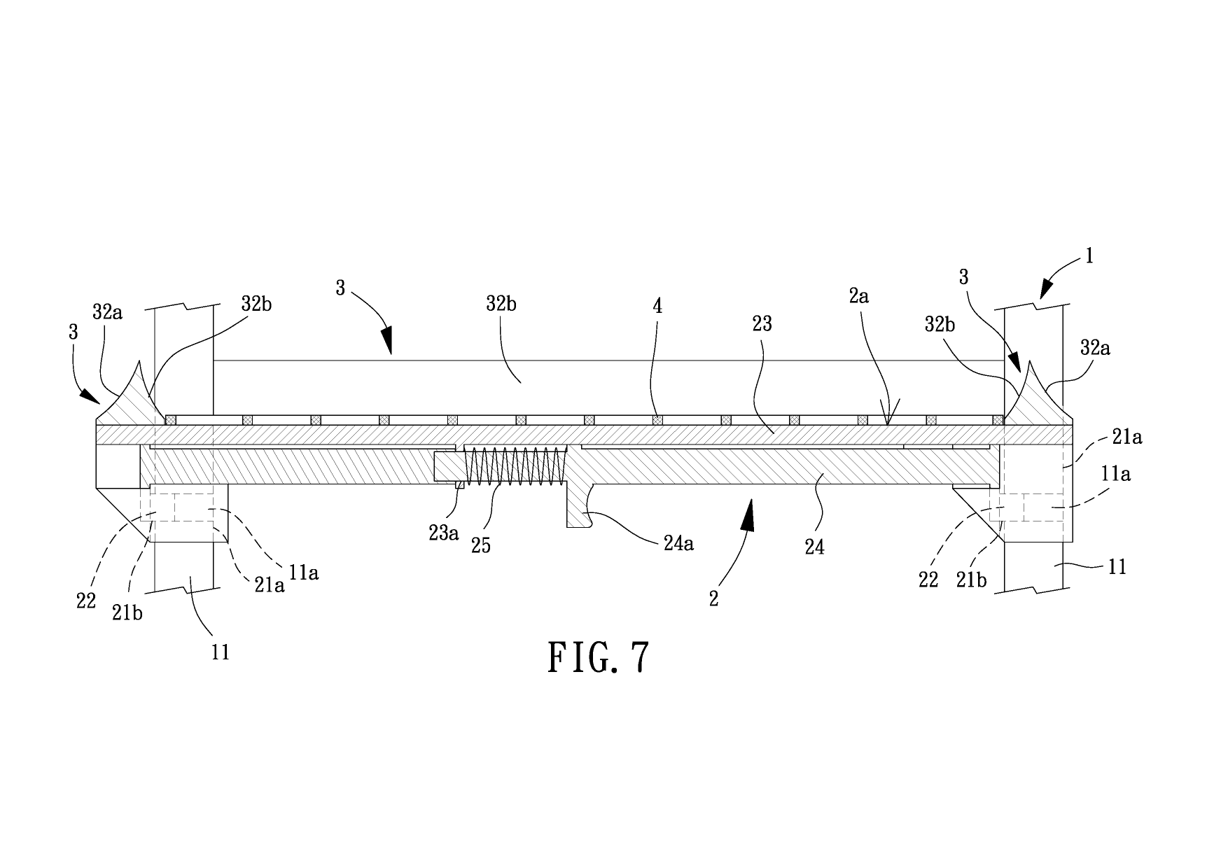

With reference to FIG. 7, the two coupling portions 22 of each sliding member 2 respectively extend through the two sliding portions 21 and are respectively inserted into two positioning portions 11a (which are at the same height) of two posts 11. Therefore, by selecting the height of two respective positioning portions 11a into which the sliding member 2 is inserted, the fixed height of the supporting faces 2a of the sliding member 2 and the adjustable board 4 can be decided.

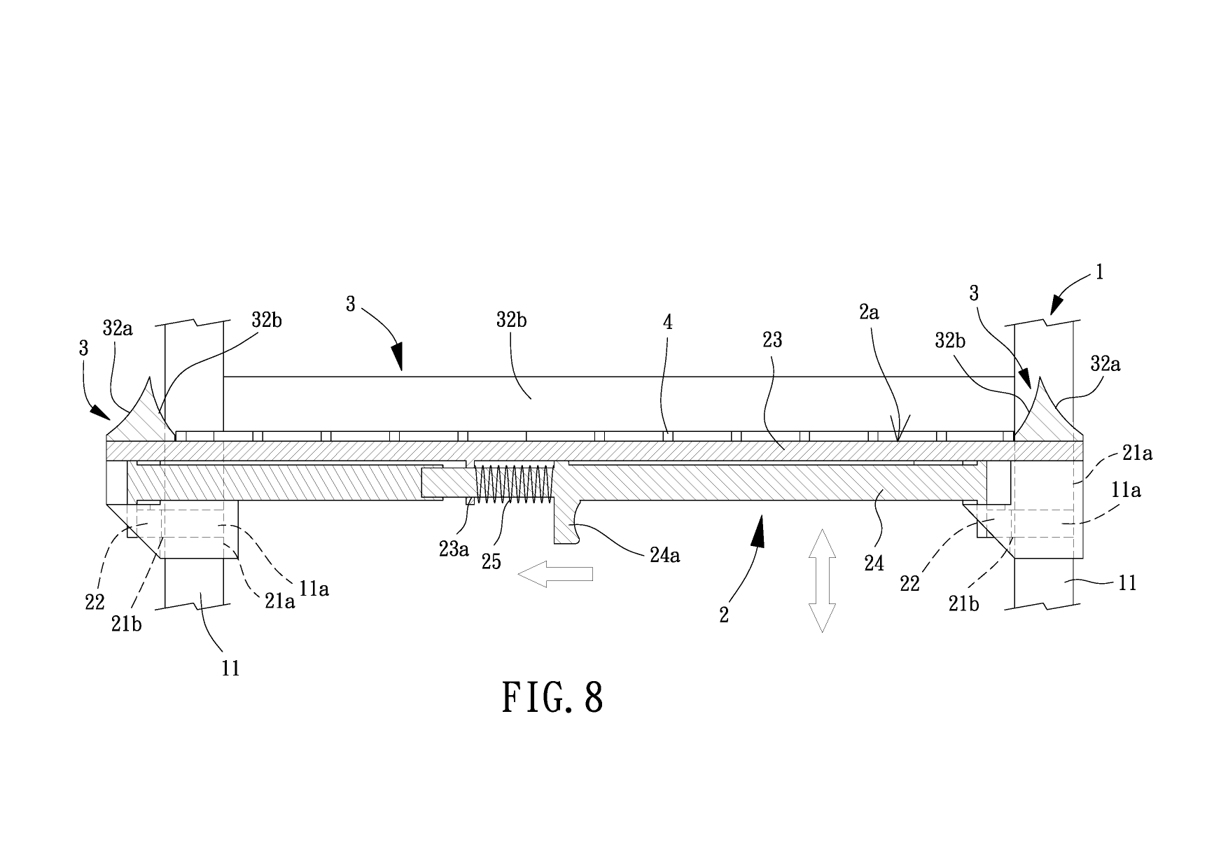

With reference to FIG. 8, by using the force application portion 24a of the pin 24 to apply a leftward force to the sliding member 2, the two coupling portions 22 are moved out of the two respective positioning portions 11a, such that the support 23 and the pin 24 may move relative to each other. At the same time, the elastic element 25 is compressed to generate a rightward returning force. Therefore, when a force is continuously applied to the force application portion 24a, the sliding member 2 may be moved vertically along the two posts 11 via the two sliding portions 21, thereby adjusting the height. Furthermore, after the height is selected, the leftward force applied to the force application portion 24a is released, and the rightward returning force of the elastic element 25 pushes the pin 24 to move rightward. Furthermore, the two coupling portions 22 are aligned with and inserted into another two positioning portions 11a at the selected height, thereby fixing the height of the sliding member 2 again.

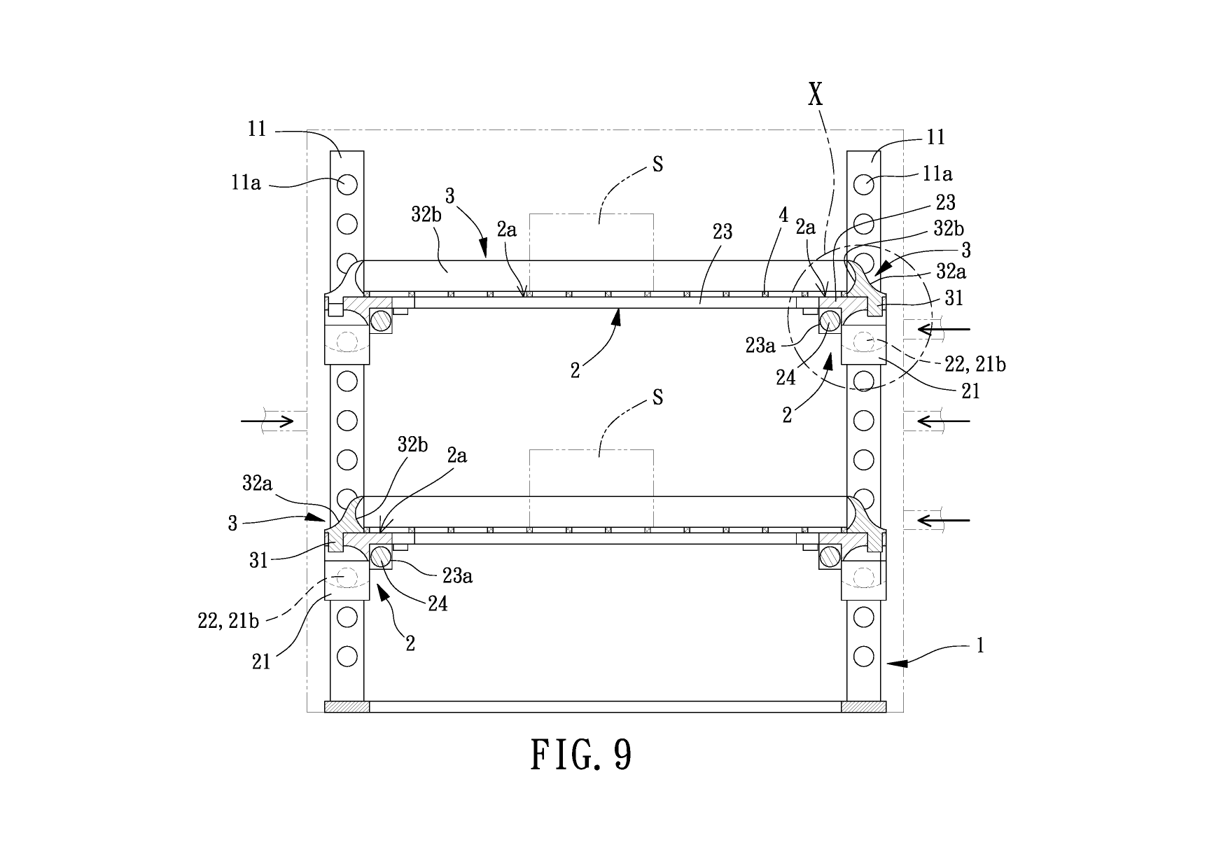

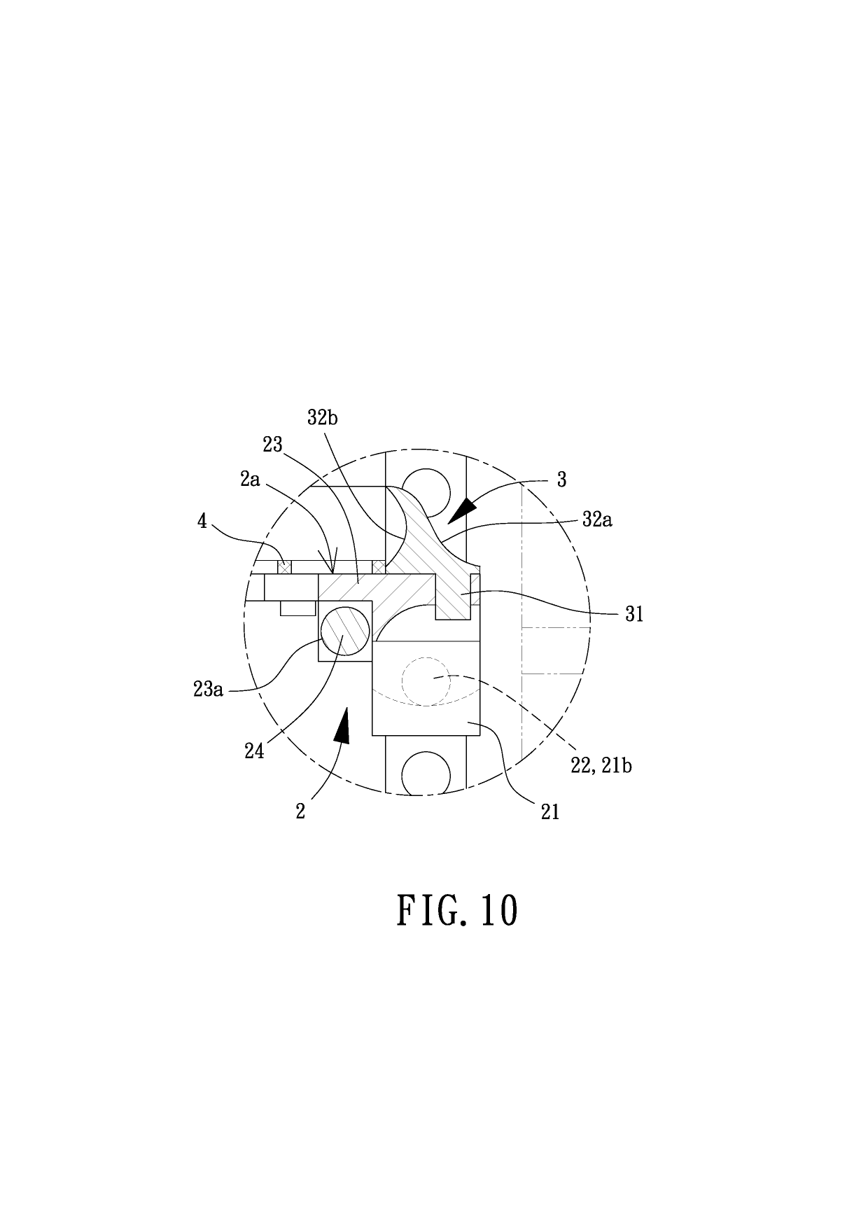

Please refer to FIGS. 9 and 10 showing another embodiment of the flow guiding members 3. As shown in FIG. 9, the cross section of each flow guiding member 3 may be in the form of a fish fin. The outwardly facing flow inducing face 32a of each flow guiding member 3 may be a wavy face, and the inwardly facing glow guiding face 32b may be a concave face. This enables circumfluence of the gas, thereby avoiding the gas from staying in the dead spaces or uneven distribution.

In summary, in the adjustable sterilizing shelf according to the present invention, the sliding members slidable in the vertical direction enables adjustment of the height of each adjustable board, such that the receiving space between two adjustable boards may be selected according to the size and the shape of the object to be sterilized. Furthermore, each adjustable board is surrounded by the plurality of flow guiding members, such that the sterilizing gas may be guided to circulate in the space between each two adjacent adjustable boards to thereby increase the distribution uniformity of the sterilizing gas.

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims. Furthermore, in a case that several of the above embodiments can be combined, the present invention includes the implementation of any combination.

What is claimed is:

1. An adjustable sterilization shelf comprising:
a frame including a plurality of posts disposed upright and around a sterilization space; and
at least one adjustable board disposed in the sterilization space, wherein the at least one adjustable board is slidable in a vertical direction and is coupled with the plurality of posts, wherein each of two opposite sides of the at least one adjustable board is provided with a flow guiding member, wherein each flow guiding member includes a flow guiding face facing the sterilization space, wherein the flow guiding face is at a first inclination angle to a surface of the at least one adjustable board, and wherein the first inclination angle is greater than 90 degrees.

2. The adjustable sterilization shelf as claimed in claim 1, wherein the at least one adjustable board is provided with at least one pair of sliding members, wherein each sliding member includes at least one sliding portion which is slidable in the vertical direction and which is coupled with a respective one of the plurality of posts, wherein each sliding member includes a supporting face, wherein the supporting faces of the at least one pair of sliding members are located on a same horizontal plane, and wherein the at least one adjustable board is located on the supporting faces of the at least one pair of sliding members.

3. The adjustable sterilization shelf as claimed in claim 2, wherein each flow guiding member is coupled with the supporting faces of the at least one pair of sliding members.

4. The adjustable sterilization shelf as claimed in claim 2, wherein a lower surface of each sliding member is provided with an auxiliary flow guiding member, wherein each auxiliary flow guiding member includes an auxiliary flow guiding face facing the sterilization space, wherein the auxiliary flow guiding face is at a second inclination angle to a lower surface of the at least one adjustable board, and wherein the second inclination angle is greater than 90 degrees.

5. The adjustable sterilization shelf as claimed in claim 2, wherein each of the plurality of posts includes a plurality of positioning portions spaced from each other in the vertical direction, and wherein each sliding member includes a coupling portion securely coupled in one of the plurality of positioning portions.

6. The adjustable sterilization shelf as claimed in claim 5, wherein each sliding member includes a support and a pin, wherein each of two ends of the support forms one of the sliding portions, wherein each sliding portion includes a receiving hole slidably receiving a respective one of the plurality of posts, wherein each of two ends of the pin forms one of the coupling portions, wherein the two coupling portions are associated with the two sliding portions, and wherein each coupling portion extends through a lateral hole of a respective one of the sliding portions and is then securely coupled with one of the plurality of positioning portions of a respective one of the plurality of posts.

7. The adjustable sterilization shelf as claimed in claim 6, wherein each sliding member is provided with an elastic element, wherein two ends of the elastic element respectively abut against a fixing portion of the support and a force application portion of the pin.

8. The adjustable sterilization shelf as claimed in claim 7, wherein the pin includes two sections assembled with each other at a junction, wherein each of two ends respectively of the two sections remote from the junction includes one of the coupling portions, wherein the two sections are engaged with the fixing portion of the support, and wherein the elastic element is held between the fixing portion of the support and the force application portion of the pin.

9. The adjustable sterilization shelf as claimed in claim 1, wherein the flow guiding face of each flow guiding member faces inward toward the sterilization space, wherein each flow guiding member further includes a flow inducing face opposite to the flow guiding face, wherein a cross section of each flow guiding member is an approximate isosceles triangle and has two sides, wherein each of the two sides is concave, and wherein the two sides respectively form the flow inducing face and the flow guiding face.

10. The adjustable sterilization shelf as claimed in claim 1, wherein the flow guiding face of each flow guiding member faces inward toward the sterilization space, wherein each flow guiding member further includes a flow inducing face opposite to the flow guiding face, wherein a cross section of each flow guiding member is in a form of a fish fin, and wherein the fish fin includes a wavy face forming the flow inducing face and a concave face forming the flow guiding face.

11. The adjustable sterilization shelf as claimed in claim 1, wherein the at least one adjustable board is in the form of a mesh board.
